(19) United States
(12) Patent Application Publication
(10) Pub. No.: US 2008/0039810 A1
(43) Pub. Date: Feb. 14, 2008

(54) Liquid absorbing cooling pad

(76) Inventors: Janet Armstrong Lee, Lilburn, GA (US); Robert Eugene Lee, Lilburn, GA (US)

Correspondence Address: LAURENCE T. PEARSON, 5503 HIGHLAND COURT, WILMINGTON, DE, 19802, US

(21) Appl. No.: 11/974,060

(22) Filed: Oct. 11, 2007

Related U.S. Application Data
(63) Continuation-in-part of application No. 11/313,093, filed on Dec. 21, 2005.

(51) Int. Cl. A61F 13/15

(52) U.S. Cl. 604/358; 604/378

(57) ABSTRACT

An article for cooling the perineum and simultaneously absorbing blood and body fluids. The article has front and rear regions. The front region serves to absorb fluid and contains a fluid permeable web and an absorbent web. The rear region is a barrier web and absorbs and prevents leakage of fluid and cooling medium from the pad. A cooling medium is disposed in between the front and rear regions.

### PRIORITY DATA

This application is a continuation in part of application Ser. No. 11/313,093, filed on Dec. 21, 2005.

### FIELD OF THE INVENTION

This invention relates to single use medical devices and more particularly to improvements in absorbent articles designed for thermal transfer.

### BACKGROUND ART

Cooling devices are known in the art. The use of ice to reduce swelling and inflammation of a wounded body area is known to assist in healing. When trauma inflicts a body part, such as a knee, swelling and inflammation of that area can occur. Inflammation is the result of the body introducing additional blood flow to the traumatized area. Additional blood flow assists in the healing of the wounded area by carrying away damaged or dead tissue. Swelling is the body's way of providing a “natural splint” to the traumatized area. Unfortunately, both inflammation and swelling can cause additional pain due to the force exerted upon the traumatized area. It is therefore advantageous to reduce the swelling and inflammation by applying a cooling device to affected area.

Devices to assist in the reduction of inflammation and swelling are known. For instance, U.S. Pat. No. 4,628,932 describes an ice pack for use on a knee. Two compartments are employed to receive the ice. This device is helpful in the reduction of inflammation and swelling to the knee but is unfortunately limited in many ways. For instance, the device is limited for use on joints such as knees and elbows and lacks the structural components to be adaptable to other body parts. Further, it lacks an outer layer which could be used to moderate the temperature, which would make the bag more comfortable. Furthermore, it lacks an absorbable material layer which could absorb body fluids that may seep from a wound. This device could be wrapped in a cloth towel. However, if any body fluids seep from the wound (i.e., blood), the cloth towel would then need to be discarded. This results in added expense of a towel and handling exposure.

U.S. Pat. No. 4,951,666 describes an ice pack wrappable about a limb. The ice pack comprises a cloth outer bag having an exterior, an interior and an open top. A sealable plastic inner bag is received within the outer bag, the inner bag having an exterior formed by opposing planar portions, an interior and a mouth opening for receiving ice, the inner bag being foldable between a retracted position wholly contained within the interior of the outer bag and an extended position in which the inner bag protrudes from the open top of the outer bag for filling.

The ice glove is a very common device used to provide cooling to the perineum. Because of its superior cooling properties, it is used in cases of extreme swelling or in the presence of a hematoma or hemorrhoids. The ice glove is made by filling an examination glove with crushed ice, tying a rubber band around the open end to reduce leakage and wrapping the glove with a cloth.

Although, the ice glove provides cooling, there are obvious problems of lack of comfort and contamination. It is difficult for the patient to sit, stand, or walk with the ice glove in place. The cloth is rough to touch, provides poor absorption and poor moderation of thermal transfer. This cooling device also creates biohazard issues during handling and laundering. Creating an unsafe environment for healthcare workers as addressed in U.S. Pat. No. 6,786,880.

The use of cold packs that do not use ice for thermal therapy is known. One type of cold pack contains a material which, upon cooling in a refrigerator or freezer, gradually warms back to ambient temperature while being applied to a wounded body part. Another type of cold pack operates via a change-of-phase of the components. Still another type of cold pack employs chemical components that are endotherm-producing on dissolution in a solvent.

Phase change materials may be converted between solid and liquid phases and utilize a latent heat of fusion to absorb, store and release heat, or cool, during such phase conversion. Accordingly, the amount of energy absorbed upon melting or released upon freezing is greater than the amount of energy absorbed or released upon increasing or decreasing the temperature of the material. Ice/water is one example of a phase change material.

Certain chemical compounds, once dissolved, result in a lowering of the temperature of the solution below ambient temperature. On dissolution, these compounds absorb heat from the surrounding environment. For example, inorganic salts or soluble organic compounds known to have a positive enthalpy of aqueous solution are used.

Examples of cold packs that employ a thermal material are those that contain a gel. Typically, these cold packs are cooled in a refrigerator or freezer. For cold therapy, once cooled, it is placed on the injured or sore area.

Chemical cold packs that provide an insulating layer between the cold pack and the skin are also known. One such cold pack uses an outer pouch containing capillaries to allow drainage of the pack and to provide a temperature moderating effect. Another chemical cold pack uses a wetting member in contact with the chemical cold pack, thereby providing moist cold to an injury. Yet another chemical cold pack positions an absorbent/insulating layer between the chemical cold and the ambient air with the chemical cold pack placed against the patient's skin. In this device, the surface area of the absorbent/insulating layer is smaller than the surface area of the cold pack. One prior art chemical cold pack cover provides means to attach the cold pack to a patient and to provide a water-resistant material in contact with the skin.

A variety of perineal cold packs consists of a feminine pad with a chemical cold pack wrapped with a thin layer of fluid-permeable non-woven fabric. The cooling is activated by squeezing the outer bag containing ammonium nitrate such that the inner bag containing water ruptures and mixes the contents. The pack is then secured to the patient's undergarment. Although the cold pack does provide adequate cooling, the blood and body fluids are not quickly absorbed. On the contrary, there is much “run-off” causing considerable amount of contamination. The other major problem with the chemical cold pack is the stiffness of the plastic bag holding the chemicals. The “crisp-feel” is not comfortable to the patient with an episiotomy or hemorrhoidectomy.

The two major complaints from the patients regarding the chemical cold packs are the mess and discomfort involved. The main concern of the medical staff is the design of the chemical cold pack results in unnecessary exposure to blood and bodily fluids.

U.S. Pat. No. 6,470,705 describes a disposable ice pack for receiving and retaining a frozen material and for compressing a traumatized area. The disposable ice pack consists of a square bag portion having four side edges, including a sealable open top end, a closed bottom end and a pair of opposed side edges forming an inner cavity. A Ziploc® type closure mechanism is along inner surfaces providing a water tight seal. A sheet material consisting of one or two layers of fluid absorbable cloth material is attached by heat sealing and juxtaposed to one of the bag portion outer walls. The fluid absorbent makes contact with the traumatized area and absorbs any body fluids. The inner cavity receives and retains the frozen material.

U.S. Pat. No. 6,251,131 describes an absorbent ice bag comprising an ice bag defining a single-side surface area, and a re-sealable opening. An absorbent article which may be hour glass shaped for use over the perineum is attached to the ice bag, the absorbent article defines an area such that the total area of the article is larger than the single side-surface area. A phase change material may be added to the ice bag through a re-sealable opening and wherein the unobstructed perimeter area provides an unobstructed area for absorbing a bodily fluid on the absorbent article. A disadvantage of the product of U.S. Pat. No. 6,251,131 patent is that there is a reduced absorbency in the region where there is ice bag.

Problems of adequate absorption and cooling have not been addressed effectively by any of the aforementioned. Requirements include a comfortable fitting shaped pad with effective absorbency and the ability to apply cold to the perineum. When ice is used as the cooling medium, the pad must remain dry.

Some inventions have attempted to add an outer layer to their respective devices. Such can be seen in U.S. Pat. Nos. 5,074,300, 5,133,348 and 5,456,704. Unfortunately, all of these inventions fall short of disclosing, teaching or suggesting, a disposable ice pack having a fluid absorbable outer layer that moderates the thermal transfer. Such a device is clearly needed to overcome all of the deficiencies of the prior art.

The present invention addresses these needs by providing for an absorbent pad that also applies cold to the perineal area and in which the absorbency of the pad is not affected by the presence of ice.

### SUMMARY OF THE INVENTION

This invention is an article for cooling the perineum and simultaneously absorbing blood and body fluid. The article comprises an essentially planar and flexible front region that contacts the body over at least a portion of the surface of the region, an essentially planar and flexible rear region that contacts the undergarment if one is being worn by the user, and is adjacent to the front region, and a cooling medium disposed in a pocket formed by the front and rear regions. The front region comprises a body facing first fluid permeable web, and a first absorbent web, the two webs being in a face to face relationship with each other. The rear region comprises a second absorbent web, and a fluid impermeable backsheet adjacent to the second absorbent web and in a face to face relationship with it. In the absence of a cooling medium, the two absorbent webs are adjacent to each other and form a pocket into which a cooling medium is inserted when the article is ready for use.

The front and rear regions are bonded around a portion of their edges leaving an unbonded portion of edge to form the pocket with an opening (gap) along a portion of the edge of the article. The first and second absorbent webs constitute the interior of the pocket and are bonded to each other around the portion of their edges not located at the opening. The first fluid permeable web is bonded to the backsheet around a portion of its edge, and may be bonded to the first absorbent web around a portion or all of the surface of the first absorbent web. The backsheet may be bonded to the second absorbent web on a portion or all of the surface of the second absorbent web. The cooling medium is situated in the pocket formed by the first and second absorbent webs. The fluid impermeable backsheet may be extended at the opening to comprise a flap that comprises a means for partially sealing the gap to contain the cooling medium in the pocket. The means for sealing the gap may be located on any of the constituents of the front and rear regions.

The first and second absorbent webs may comprise non-wovens and may also comprise superabsorbent polymer.

The body facing first fluid permeable web may comprise a non-woven or a formed film.

The fluid impermeable backsheet may comprise a film, or a non-woven laminated to a film, with the non-woven being laminated on the surface of the film outside the pad.

In a further embodiment of the invention, the means for sealing is selected from a group consisting of a press and seal fastener like those sold under the trade name Ziploc®, hook and catch fasteners like those sold under the trade name Velcro®, and a pressure sensitive adhesive double sided tape.

In a still further embodiment of the invention, the first absorbent web, or the second absorbent web, or both are joined to the body facing first fluid permeable web, or the fluid impermeable backsheet by their edges or any portion of their surfaces.

The fluid impermeable backsheet can also comprise adhesive on all or a portion of its external surface, the external surface being that which is designed to adhere to an undergarment.

The invention is also directed towards a method for cooling a wound while absorbing body fluids. The method comprises the step of applying the cooling and absorbent pad described above to the wound.

### BRIEF DESCRIPTION OF THE DRAWINGS

FIG. 1 shows an exploded view of the layer arrangement of an embodiment of the product of the invention.

FIG. 2 shows a cross section of an assembled version of the pad of the invention.

FIG. 3 shows a view of the outside of the invention away from the body side.

### DETAILED DESCRIPTION OF THE INVENTION

### Definitions

The term “film” refers to a web made by extruding a molten sheet of thermoplastic polymeric material by a cast or blown extrusion process and then cooling the sheet to form a solid polymeric web. Films can be monolayer films, coextruded films, coated films, and composite films. Coated films are films comprising a monolayer or coextruded film which are subsequently coated (for example, extrusion coated, impression coated, or printed) with a thin layer of the same or different material to which it is bonded and after bonding is incapable of separation. Composite films are films comprising more than one film where at least two films are combined in a bonding process. Bonding processes may incorporate adhesive layers between the film layers. Films also denote cast films that are not made using an extrusion process.

Throughout this description, the expression “apertured film” denotes a film having a plurality of holes that extend from one surface to a second surface. A two dimensional apertured film is a film in which no three dimensional structure exists in or around the holes, which then connect the second surface of a flat film to the first surface of the film. A three dimensional film is a film with protuberances or other dimensional structures.

By “formed film” is meant films made by forming two-dimensional sheets to create three-dimensional formed films. It is known to vacuum-form two-dimensional sheets to create three-dimensional formed films, by causing a softened or molten polymer sheet to conform to the shape of a forming screen. Commonly utilized apertured formed film topsheets are disclosed in, for example U.S. Pat. Nos. 3,929,135, 4,324,246, 4,342,314, 4,252,516, 4,535,020, 5,591,510, and U.S. patent application Ser. No. 10/082,040 filed Feb. 20, 2002. The formed films described in these publications are formed by application of a fluid pressure differential where the fluid preferably is air to thereby cause the polymer sheet to conform to the screen and also to cool. A preferred feature of these formed films is that the polymer crystallizes, or “sets” after the molten polymer has been formed. The resultant formed film therefore is said to have “memory.” Memory enables the formed film to regain its original form after non-deforming forces are applied to it, for example while the film is stored in the form of a wound roll. A disadvantage of vacuum formed films is that the body contacting surface of the film my have a plastic feeling that can feel both sticky and hot to the wearer of the article.

It also is known to utilize high pressure water jet systems, known as hydroforming systems, to cause two-dimensional polymeric films to conform to forming screens in order to create three-dimensional films. U.S. Pat. Nos. 4,609,518, 4,629,643, 4,695,422, 4,778,644, and 4,839,216, all to Curro (referred to herein as “Curro”) and assigned to Procter & Gamble, and U.S. Pat. Nos. 5,770,144, 6,022,607, and 6,240,817 assigned to McNeil, describe a multitude of film designs created by the combination of forming screens using high pressure water jet systems. In these systems, pressurized water is ejected from nozzles projecting water onto the surface of a polymeric film causing it to conform to the surface of an underlying support screen. The screen has indentations or perforations disposed on its surface. As the screen rotates it causes subsequent portions of the film to pass under the water jet causing subsequent portions of the film to conform to the screen. An advantage of this method of making formed films is that the number of protrusions that can be made in a specific surface area of film is greater than what is possible in a vacuum formed film process. Another advantage is that by utilizing water to deform crystalline film high levels of energy are applied to the film thereby causing more work and making the film softer.

The term “web” refers to a material capable of being wound into a roll. Webs can be film webs, non-woven webs, woven webs, laminate webs, apertured laminate webs etc.

The term “non-woven” means a web including a multitude of fibers. The fibers can be bonded to each other or can be unbonded. The fibers can be staple fibers or continuous fibers. The fibers can comprise a single material or a multitude of materials, either as a combination of different fibers or as a combination of similar fibers each comprised of different materials.

A non-woven fibrous web useful in embodiments of the invention may comprise fibers of polyethylene, polypropylene, elastomers, polyesters, rayon, cellulose, nylon, and blends of such fibers. A number of definitions have been proposed for non-woven fibrous webs. The fibers usually include staple fibers or continuous filaments. As used herein “non-woven fibrous web” is used in its generic sense to define a generally planar structure that is relatively flat, flexible and porous, and is composed of staple fibers or continuous filaments. For a detailed description of non-wovens, see “Non-woven Fabric Primer and Reference Sampler” by E. A. Vaughn, ASSOCIATION OF THE NON-WOVEN FABRICS INDUSTRY, 3d Edition (1992). The non-wovens may be carded, spun bonded, wet laid, air laid and melt blown as such products are well known in the trade.

The non-woven web used in the present invention can be the product of any process for forming the same. Examples of methods for manufacturing non woven webs that are well known to those skilled in the art are the processes that produce spunbond and melt blown non woven webs. The non woven web of which embodiments of this invention comprise can also be a composite or combination of webs, such as spunbond or melt blown webs. In a preferred embodiment of the invention, the web is a spunbond material made of polypropylene fiber. However, the non woven web can comprise any polymeric material from which a fiber can be produced.

The term “essentially planar” refers to an article that has a flat surface of the form of a flexible web or sheet which may be bent or folded into shape to fit a patient's body.

“Superabsorbent polymers” refers to synthetic cross-linked polymeric materials that are capable of absorbing many times their own weight in water and other liquids. As used herein, the term “SAP” means a superabsorbent polymer which, when in a substantially. dry state, has the ability to spontaneously imbibe more than twenty times its own weight in aqueous fluid, for example, tap water. Any super absorbent polymer (SAP) can be used in the present invention. Usually, the SAP component is a cellulose-derived particle, polyacrylic acid based material, and the like. Typically, SAP is produced in granular form, such granules exhibiting a particle size distribution and an average particle size distribution. The average particle size distribution should be related to the un-stretched thickness of the film in such way that the largest particles fit within the thickness of the un-stretched films. Particles produced in larger than adequate sizes may be ground down to the appropriate size, if needed.

By “means for sealing” is meant the means by which a pouch or bag that the invention comprises for holding a cooling medium can be sealed around all or a portion of its edge to make if fluid tight. Examples of means for sealing are zipper like structures such as Ziploc® (S. C. Johnson, Racine, Wis.), hook and loop type structures such as Velcro® (Velcro Industries B.V., Curacao, NL), or pressure sensitive adhesive on the surfaces of the pad, said adhesive sealing when pressed with the force applied by a human hand. For example, Velcro® can be applied to two surfaces to be sealed or to a flap on one surface that folds over to attach to a Velcro® strip attached to a second surface.

In one embodiment of the invention the sides of the pad are elasticized to ensure a closer fit to the woman's body. Any elastic material maybe used, but preferably the material will be either a metallocene based low density polyethylene (m-LDPE), or a block-copolymer blend that contains styrene/butadiene/styrene (SBS), styrene/ethylene-butylene/styrene (SEBS), ethylene vinyl acetate (EVA), thermoplastic urethane, or cross-linked rubber. Desirably, the elastic polymeric film has a basis weight of from about 18 g/m² to about 100 g/m². Preferably, an m-LDPE film has a basis weight of about 25 g/m², whereas block copolymer films have a basis weight of about 50 g/m². Also, it is desirable that the elastic polymeric files have less than 25% set when stretched 50%.

Elastication can be accomplished by making one of the layers of the pad of elastic material, or by attaching an elastic strip to the edge of the pad by adhesive or some other means for attachment known to one skilled in the art.

By “cooling medium” is meant any solid or fluid that has sufficient heat capacity to be used in the product of the invention to take heat form the patient's body. In one embodiment of the invention the preferred cooling medium is ice, due to its availability and low cost. The use of cold packs that do not use ice for thermal therapy is known and one skilled in the art will be able to select a suitable cooling medium for use in the invention.

One type of cooling medium contains a material which, upon cooling in a refrigerator or freezer, gradually warms back to ambient temperature while being applied to a wounded body part. Another type of cold pack operates via a change of phase of the components of the cold pack. Still another type of cold pack employs chemical components that are endotherm-producing on dissolution in a solvent.

Phase change materials as cooling media may be converted between solid and liquid phases and utilize a latent heat of fusion to absorb, store and release heat, or cool, during such phase conversion. Accordingly, the amount of energy absorbed upon melting or released upon freezing is greater than the amount of energy absorbed or released upon increasing or decreasing the temperature of the material. Ice/water is one example of a phase change material.

Chemical compounds that, once dissolved into a solution, result in a lowering of the temperature of the solution below ambient temperature. On dissolution, these compounds absorb heat from the surrounding environment. For example, inorganic salts or soluble organic compounds known to have a positive enthalpy of aqueous solution are used to make the reduced temperature solutions useful as cooling media.

Examples of cold packs that employ an insulating material are cold packs that contain a gel. Typically, these cold packs are cooled in a refrigerator or freezer. For cold therapy, once cooled, the cold pack is placed on the injured or sore area and thus provides the cold therapy.

The article can be assembled by any means known to one skilled in the art. The edges of selected sheets that the pad comprises are adhered to each other around a margin adjacent to the edges of the sheets. Means for adhesion can include hot melt or solvent based adhesives. The edges can also be stitched. In non-limiting examples, polymers that have been used in hot melt adhesives employed in the construction of disposable non-woven goods have included S-I-S (styrene-isoprene-styrene); SBS (styrene-butadiene-styrene); SEBS (styrene-ethylene-butylene-styrene); EVA (ethylene vinyl acetate); and APAO (amorphous poly alpha olefin).

### Preferred Embodiments

This invention is an article for cooling the perineum and simultaneously absorbing blood and body fluid. The article comprises an essentially planar and flexible front region that contacts the body over at least a portion of the surface of the region, an essentially planar and flexible rear region that contacts the undergarment if one is being worn by the user, and is adjacent to the front region, and a cooling medium disposed in a pocket formed by the front and rear regions. The front region comprises a body facing first fluid permeable web, and a first absorbent web, the two webs being in a face to face relationship with each other. The rear region comprises a second absorbent web, and a fluid impermeable backsheet adjacent to the second absorbent web and in a face to face relationship with it. In the absence of a cooling medium, the two absorbent webs are adjacent to each other, bonded around a portion of their edges, and form a pocket into which the cooling medium is inserted when the article is ready for use.

The front and rear regions are bonded around a portion of their edges leaving an unbonded portion of edge to form the pocket with an opening (gap) along a portion of the edge of the article. The first and second absorbent webs constitute the interior of the pocket and are bonded to each other around the portion of their edges not located at the opening. The first fluid permeable web is bonded to the backsheet around a portion of its edge, and may be bonded to the first absorbent web around a portion or all of the surface of the first absorbent web. The backsheet may be bonded to the second absorbent web on a portion or all of the surface of the second absorbent web. The cooling medium is situated in the pocket formed by the first and second absorbent webs. The fluid impermeable backsheet may be extended at the opening to comprise a flap that comprises a means for partially sealing the gap to contain the cooling medium in the pocket. The means for sealing the gap may be located on any of the constituents of the front and rear regions.

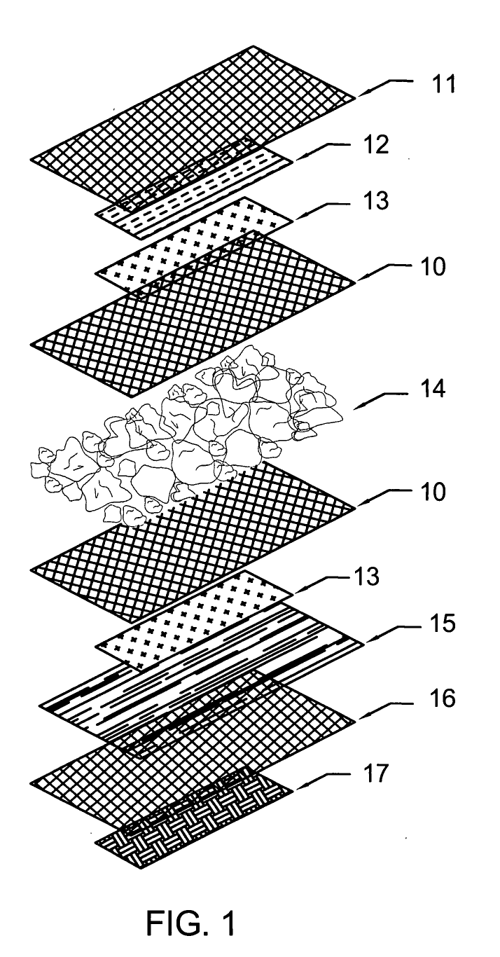

The invention can be further understood by reference to the figures. In FIG. 1 is shown an exploded view of one embodiment of the pad of the invention that shows the order in which the layers can be assembled. A cooling medium (14), such as ice, is disposed in a pocket bounded by two absorbent web layers (10). The absorbent webs can independently be any material that absorbs fluid. For example, either absorbent web can be a fluffy batt cut from a relatively loose web of non-woven fibers having a relatively high absorptive capacity. The absorbent webs usually have a rectangular configuration, and may optionally have inwardly curved side edges, such as an hourglass shape. The absorbent web is usually smaller in surface area than the backsheet (15). The absorbent core may also be a fibrous batt having an integral densified layer. The absorbent layers can also be a non-woven, and their absorbency can be supplemented by the superabsorbent material (13). Although the figures show the superabsorbent material as separate layers (13), the superabsorbent can also be distributed into the absorbent layer (10) on either side of the cooling medium (14), for example as crystals. In a further embodiment of the invention, the superabsorbent material may also be placed only on one side of the cooling medium (14).

The absorbent web may contain any material that absorbs bodily secretions including, but not limited to pulp, polymeric fibers and filaments, spagnum moss, natural fibers, superabsorbent polymers (including fibers, particulate material and foams), absorbent foams, and other such absorbent materials. The absorbent core may also include additional materials such as odor control material, wetness indicator material, materials for administering or delivering medications, such as encapsulated medications, and materials for maintaining skin moisture, such as encapsulated moisturizers.

On the body side of the pad, a topsheet (11), optional distribution layer (12) and one absorbent layer (10) with optional superabsorbent material (13) on the body side of the pad form a front region of the pad. Fluid is captured by the topsheet (11) that may be a non-woven or a formed film. The topsheet is in fluid contact with an optional distribution layer (12) that can also be a non-woven or a formed film. The distribution layer serves to spread the captured fluid in the plane of the pad so as to avoid local saturation of the absorbent materials (10 and 13). A non limiting example of such a layer is found in U.S. Pat. No. 6,700,036 to Tredegar.

On the outside layers of the pad the backsheet (15), optional non-woven layer (16) and optional adhesive layer (17) together with an absorbent layer (10), and optional superabsorbent material (13) form a rear region of the pad. The absorbent layer (10) on the outside side of the pad is bounded by a non permeable backsheet (15) that prevents fluid leakage from the back of the pad. The impermeable backsheet (15) may be of any flexible material that prevents the transfer of fluid through it, but does not necessarily prevent the passages of gases. Sheets that are pervious to vapor are known as breathable sheets. In general, these backsheets are intended to allow the passage of vapor through them while retarding, at least to a degree, the passage of fluid. Porous film technology provides materials that can be used to form sheets that allow vapor transmission, but are relatively impervious to liquids. Commonly used materials are polyethylene or polypropylene films. Other materials that may be used as impermeable barriers may be chosen from films of polyesters, polyamides, polyethylene vinyl acetate, polyvinyl chloride, and polyvinylidene chloride. Co-extruded and laminated combinations of the foregoing, wherein such combinations are permitted by the chemical and physical properties of the film, may be used. Fluid impermeable nonreticulated foams and repellent treated papers may also be used. Films that are fluid barriers, but permit gases to transpire, i.e., “breathable films,” may also be used.

The further non-woven layer (16) bonded to the backsheet is optional if the pad requires a soft feel. Typically, the non-woven layer (16) is secured to the backsheet (15) prior to the assembly of the pad by a lamination process that will be familiar to one skilled in the art, for example adhesive lamination, thermal lamination, ultrasonic lamination or vacuum lamination are all methods for bonding the two layers. An adhesive layer (17) is optional if the pad is to be secured to an undergarment.

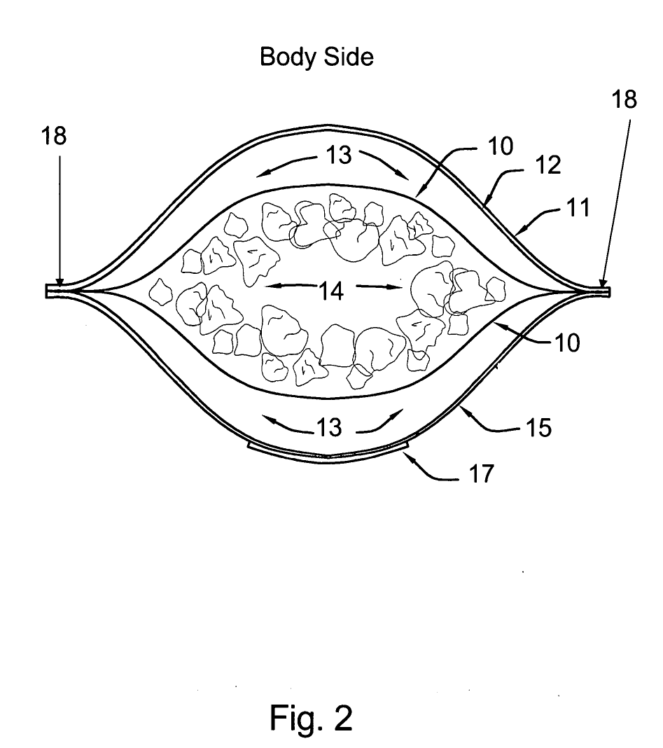

FIG. 2 shows a cross sectional view of an embodiment of an assembled pad. The pocket for the ice (14) is formed by the absorbent layers (10) which are in turn bonded at their edges to each other and optionally to the topsheet (11), distribution layer (12), and backsheet (15) and optional adhesive (17). Further absorbent or superabsorbent material (13) may be deposited in the front and rear regions of the pad. The seal (18) that is formed by the bonded edges may be produced by any means known to one skilled in the art. For example the edges may be sewn together, or glued with a hot melt or pressure sensitive adhesive. In a further embodiment, not shown in the figure, the edges of the absorbent layers (10) need not be joined to the seal (18) but are separately bonded to form a pocket into which the cooling medium is placed.

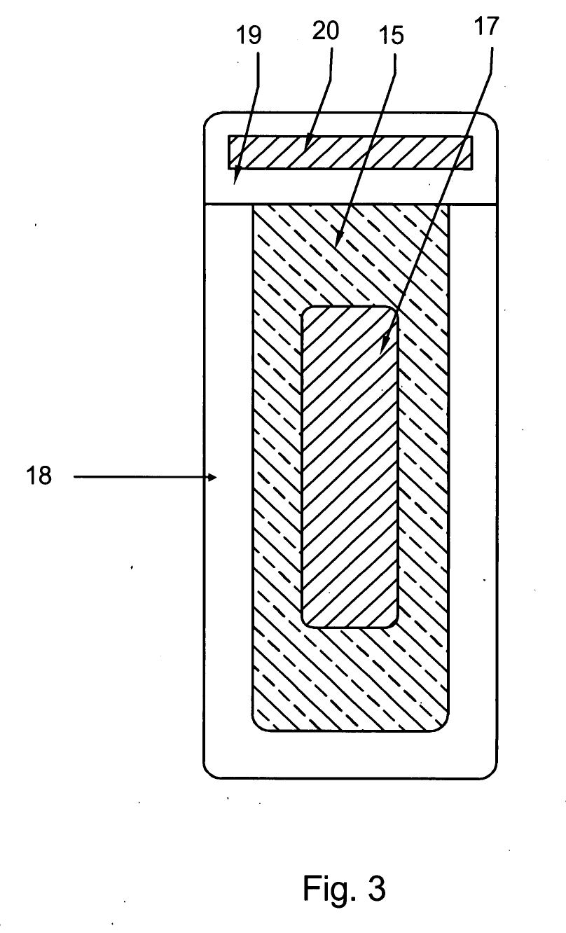

FIG. 3 shows a diagrammatic representation of a side view of an embodiment of the pad looking from the back side. An adhesive region (17) is applied to a backsheet (15). The backsheet is sealed at region (18) to the topsheet, non-woven layers, and distribution layer (not shown on the figure). The seal (18) covers three edges of the pad and a region (19) forms an opening through which a cooling medium such as ice can be placed into the pocket formed by the absorbent layers. Preferably the opening formed by the sealing of the absorbent layers is collocated with the opening formed by the sealing of the topsheet and backsheet for ease of introduction of cooling medium. The opening can be closed using a means for sealing (20). The means for sealing can be located on the backsheet, fluid permeable web, or on the first or second absorbent webs. In an alternative embodiment of the invention there need not be an adhesive layer (17) applied to the backsheet (15).

### EXAMPLE

A 15 cm by 40 cm pre-pad was constructed with a perforated spunbond fluid permeable topsheet region comprising 50% polyethylene sheath/50% polypropylene core bicomponent fibers having a fiber denier from about 3-5. (BBA, Simpsonville, S.C.), super absorbent polymer (approximately 10 g, polyacrylic acid BASF, Charlotteville, N.C.), and approximately 70 g of cellulose absorbent fluff, and a back sheet region consisting of polyethylene film laminated to a polypropylene non-woven. (Clopay, Cincinnati, Ohio). The topsheet and backsheet were stitched around the edges to form a seal.

A 20 grams per square meter (gsm) polypropylene spunbond material (Oxco, Charlotte N.C.) was formed into a pocket by folding into a 30 cm by 40 cm sheet and sealing two sides with a surger. One short side of the pocket was left open. The pocket was inserted into an opening made one of the short edges of pre-pad by pulling apart the end seam on the pre-pad. Ice (250 grams) was inserted into the pocket. The pocket was then sealed with a piece of double sided adhesive tape, size 2 cm by 10 cm.

Pads were placed on 10 patients each in areas of pain following childbirth or hemorrhoidectomy. As comparative examples, a latex glove packed with 200 g of ice, or a Cardinal Peri-Pad (Covidien, Mansfiled, Mass.) were used. No leakage of water either onto the patient or from the seams of the pad was seen from the pad of the invention during use. All patients reported enhanced comfort and lessening of pain perceived as a result of use of the pad of the invention relative to the controls.

Although the present invention has been described with respect to various specific embodiments and the example, various modifications will be apparent from the present disclosure and are intended to be within the scope of the following claims.

We claim:

1. An article for cooling a wound and simultaneously absorbing blood and body fluids, said article comprising an essentially planar and flexible front region that contacts the patient's body over at least a portion of the surface of the region, an essentially planar and flexible rear region adjacent to and in a face to face relationship with the front region, and a cooling medium disposed between the front and rear regions, said front region comprising;
(i) a body facing fluid permeable web, and
(ii) a first absorbent web in a face to face relationship with the first fluid permeable web,
and said rear region comprising;
(iii) a second absorbent web, and
(iv) a fluid impermeable backsheet adjacent to the second absorbent web in a face to face relationship with the second absorbent web,
in which
(a) the first and second absorbent webs are adjacent to the cooling medium and are sealed around a portion of their periphery so that the first and second absorbent webs form a pocket such that a gap is left over a section of their periphery, and the cooling medium is inserted into the unsealed gap and is in contact with the first and second absorbent webs, and
(b) the body facing fluid permeable web and fluid impermeable backsheet are bonded to each other around a portion of their periphery to form a pocket in which the first and second absorbent webs, and the cooling medium, reside,
the article further comprising a means for sealing the gap to contain the cooling medium in the pocket.

2. The article of claim 1 in which the first or second absorbent webs comprise non-wovens.

3. The article of claim 1 in which the first or second absorbent webs comprise superabsorbent polymer.

4. The article of claim 1 in which the body facing first fluid permeable web is a non-woven.

5. The article of claim 1 in which the body facing first fluid permeable web is a formed film or an apertured film.

6. The article of claim 1 in which the front region further comprises a distribution layer disposed in between the first fluid permeable web and the first absorbent web.

7. The article of claim 1 in which the fluid impermeable backsheet comprises a non-woven laminated to a film, with the non-woven being disposed on the surface of the film outside the article and away from the body side of the article.

8. The article of claim 1 in which the fluid impermeable backsheet further comprises adhesive disposed on the surface of the backsheet outside the article and away from the body side of the article.

9. The article of claim 1 in which the first absorbent web is joined to the body facing first fluid permeable web, by any portion of their edges or any portion of their surfaces.

10. The article of claim 1 in which the second absorbent web is joined to the fluid impermeable backsheet, by any portion of their edges or any portion of their surfaces

11. A method for cooling a wound while absorbing body fluids comprising the step of applying a cooling and absorbent pad to the wound, said pad comprising an essentially planar and flexible front region that contacts the patient's body over at least a portion of its surface, an essentially planar and flexible rear region adjacent to and in a face to face relationship with the front region, and a cooling medium disposed between the front and rear regions, said front region comprising;
(i) a body facing fluid permeable web, and
(ii) a first absorbent web in a face to face relationship with the first fluid permeable web,
and said rear region comprising;
(iii) a second absorbent web, and
(iv) a fluid impermeable backsheet adjacent to the second absorbent web in a face to face relationship with the second absorbent web,
in which
(a) the first and second absorbent webs are adjacent to the cooling medium and are sealed around a portion of their periphery so that the first and second absorbent webs form a pocket such that a gap is left over a section of their periphery, and the cooling medium is inserted into the unsealed gap and is in contact with the first and second absorbent webs, and
(b) the body facing fluid permeable web and fluid impermeable backsheet are bonded to each other around a portion of their periphery,
the pad further comprising a means for sealing the gap to contain the cooling medium in the pocket.

12. The pad of claim 11 in which the first absorbent web is joined to the body facing first fluid permeable web, by any portion of their edges or any portion of their surfaces.

13. The pad of claim 11 in which the second absorbent web is joined to the fluid impermeable backsheet, by any portion of their edges or any portion of their surfaces
